(19) United States
(12) Patent Application Publication
(10) Pub. No.: US 2007/0106193 A1
(43) Pub. Date: May 10, 2007

(54) Framework for enabling traction to be applied to a patient

(76) Inventors: Kenneth P. Davis, High Wycombe (GB); David L. Mollon, Crowthorne (GB)

Correspondence Address: RENNER, KENNER, GREIVE, BOBAK, TAYLOR & WEBER, FIRST NATIONAL TOWER FOURTH FLOOR, 106 S. MAIN STREET, AKRON, OH, 44308, US

(21) Appl. No.: 11/267,481

(22) Filed: Nov. 5, 2005

(51) Int. Cl. A61F 5/00

(52) U.S. Cl. 602/32

(57) ABSTRACT

A framework for enabling traction to be applied to a patient comprises a main elongate structure (12) supporting a pulley (15), and at least one leg structure (10) foldably connected to the main structure. Preferably, the main structure includes a foldable second leg portion (123), and a pulley support section (122) of the main structure is slidably located, in order to permit adjustment of the location of the pulley. An further support member (20) may be provided, removably attachable to the leg structure (10), for supporting bed covers.

The present invention relates to a framework which permits traction to be applied to a patient.

Such frameworks are already well known and in particular a framework known as a Braun frame has been particularly popular.

The problem with the Braun frame is that it is a heavy fixed framework which mitigates against it being moved easily from patient to patient.

The present invention provides a collapsible framework which will carry out the same functions as the known Braun frame but in a more convenient fashion.

Preferably, the framework is not only collapsible but also adjustable so as to enable it to be utilised in different locations and with different sizes of patients.

In order that the present invention be more readily understood, an embodiment thereof will now be described by way of example only with reference to the accompanying drawings, in which:

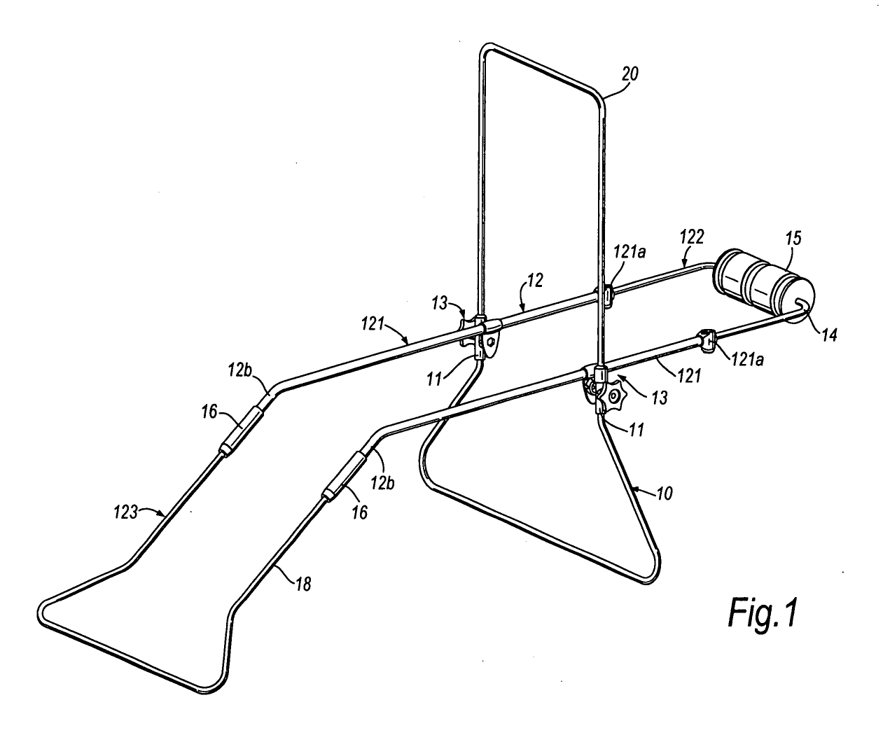

FIG. 1 shows an isometric view of a frame according to the present invention in an upright assembled position;

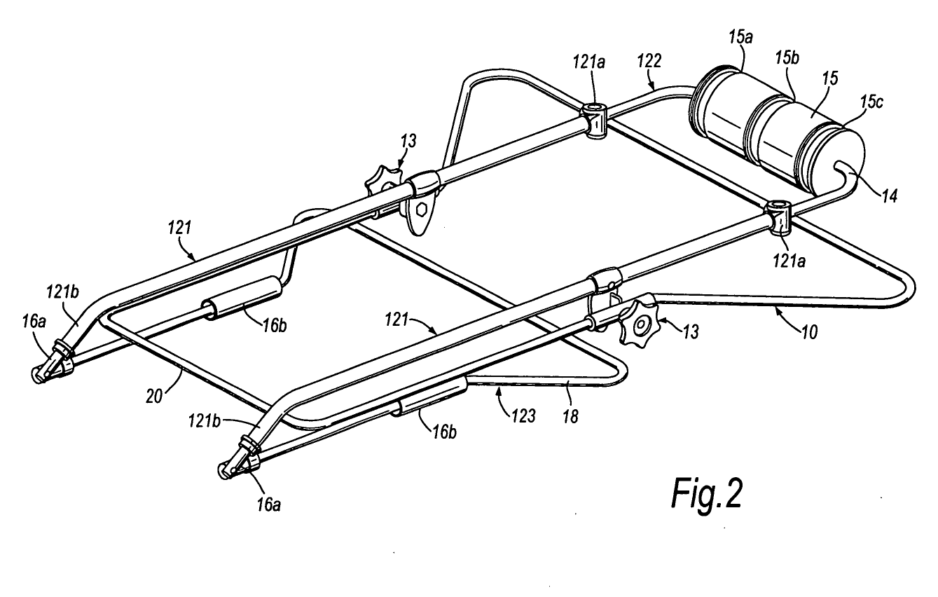

FIG. 2 shows an isometric review of the frame shown in FIG. 1 but in a folded flat condition;

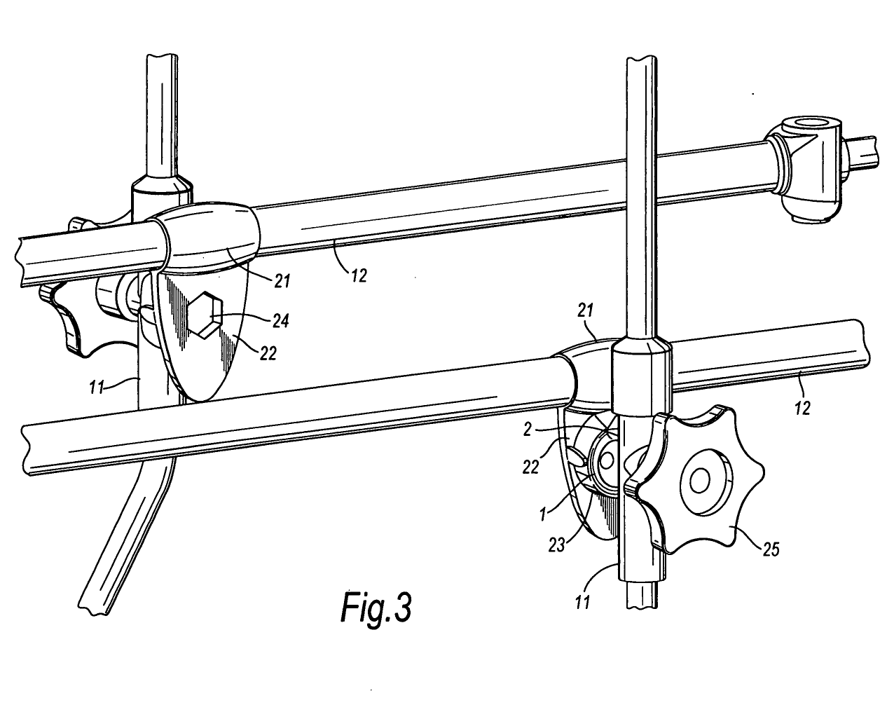

FIG. 3 shows a detail of the locking mechanism of the frame shown in FIG. 1; and

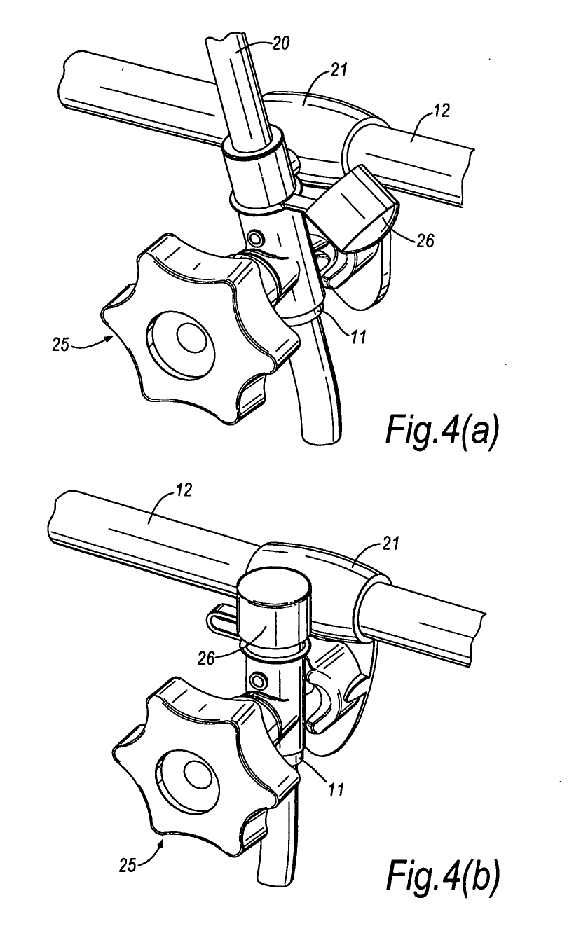

FIG. 4(a) shows a detail of the attachment of a bed cover support member to the stand member, and FIG. 4(b) shows the same part but without the bed cover support attached.

The frame for permitting traction to be applied to a patient according to the present invention comprises a plurality of interlinked members. In this case there are basically two interlinked members so as to permit folding with one of the members being further adjustable to provide a considerable degree of adjustment as well as enabling the frame to be folded in a very compact fashion. The two members are a main stand member 10 which is of generally flattened U-shape with the free ends of the legs 11 of the member 10 being connected to a main elongate member 12 via an adjustable locking linkages 13 whereby the member 10 may be folded with respect to the member 12 to assume the position shown in FIG. 2.

The member 12 in this embodiment is made up of three sections, namely a middle section 121, a pulley support section 122 and a bed engaging section 123.

The middle section 121 in this embodiment is provided with the linkages 13 connecting the two members 10 and 12 together. One end 121a of the middle section 121 is connected to the pulley support section 122 which is generally U-shaped with a pulley 15 rotatably mounted in the bight 14 of the section 122.

The U-shaped section 122 is slidably received within tubular members which form the section 121 and can be fixed any one of a number of different discrete extended positions by means of a locking adjustment arrangement cooperating with indentations spaced apart along the length of the legs of the member 122. The adjustment arrangement is preferably a simple spring loaded plunger which is biased into engagement with an indentation to fix the section 122 in position. Any other simple, easily operable system could be used.

The other ends 121b of the middle section 121 are provided with hinge arrangements 16 which connect to the bed engaging section 123.

As better shown in FIG. 2, the hinge arrangements 16 consists of a hinge part 16a and a locking tubular member 16b which can be moved from the position shown in FIG. 2 to the position shown in FIG. 1 in order to completely cover the hinge portion 16a and thus lock the bed engaging section 123 into position as shown in FIG. 1. In order to fold the section 123 to the position shown in FIG. 2 all that is necessary is for the tubular member 16b to be slipped off the hinge portion 16a whereupon the section 123 can be folded against the tubular members forming the middle section 121.

It will be noted that the tubular members forming the middle section 121 are slightly bent adjacent their ends 121b so as to permit the frame, when assembled as shown in FIG. 1 to be supported on a flattened sections of a U-shaped members 18 which constitutes the bed engaging section 123 in this embodiment and the member 10.

If necessary, a further U-shaped member 20 can be added which may fit into the free ends 11 of the flattened U-shaped member 10. The member 20 can be used to support bed covers and aid comfort. FIG. 4(a) shows in more detail the attachment of the U-shaped bed cover or sheet support member 20 to the stand member 10, and also shows a dust cap 26 which is preferably provided, and may be retained as shown by a portion surrounding the leg 11 of the stand member 10. In the configuration of FIG. 4(a), the dust cap is not used, since the tubular legs 11 of the member 10 are occupied by the member 20. However, when the bed cover support member 20 is not fitted, the dust cap 26 can be placed over the open end of each leg 11, to prevent dust or other debris from entering the tubular frame. It will be appreciated that a dust cap is preferably provided on each side of the frame.

Turning now to FIG. 3, this shows in more detail the locking arrangement between the flattened U-shaped member 10 and the main elongate member 12. This locking arrangement comprises a main part 21 which is fixed to a respective tubular member 12 and has a depending portion 22, one major surface of which is provided with a boss 23 through the centre of which passes a locking screw 24. Each free end of the legs 11 has a diametrically opposite pair of holes in register with the hole through the boss 23 so that the locking screw projects from the boss 23 through the holes in the walls of legs 11 so that a locking nut or handwheel 25 can be provided on the end of the locking screw 24. Consequently, simply by slackening the locking nuts on the locking screws 24, the U-shaped member 10 can be folded against the member 12 to adopt the position shown in FIG. 2 and the member 20, if provided, adopts the position shown in FIG. 2 also. Preferably, the locking nut or handwheel is retained in position so that it cannot be fully removed, but can only be tightened or loosened.

It is also to be noted that the boss 23 is shaped to have two pairs of diametrically opposed scallops 1, 2 so that the member 10 and main member 12 can only adopt one of two specific orientations. This not only ensures accurate positioning of the respective members but also ensures that the integrity of the framework is assured.

It is also to be noted that the pulley 15 (FIG. 2) is elongate in an axial direction so as to provide three quite distinct pulley surfaces 15a, 15b and 15c. This permits traction to be applied down the centre line of the frame by using the pulley surface 15b or slightly off axis by using the pulley surface 15a or 15c.

It will be appreciated that the above embodiment is fully adjustable to permit different sizes of patient. Further, because it is foldable it can be moved readily from patient to patient, and requires little storage space when not in use.

1. A framework for enabling traction to be applied to a patient, the framework comprising a main elongate structure (12) supporting a pulley (15), and at least one leg structure (10) foldably connected to the main structure (12).

2. The framework as claimed in claim 1, wherein the main elongate structure (12) includes a foldable second leg portion (123).

3. The framework as claimed in claim 2, wherein the second leg portion (123) is foldably connected to a middle section (121) of the main structure (12) by means of at least one lockable hinge.

4. The framework as claimed in claim 3, wherein the lockable hinge comprises a hinge portion (16a) and a substantially tubular locking member (16b) which is slidable over the hinge portion (16a) to lock the hinge in position.

5. The framework as claimed in claim 1, wherein the main structure (12) comprises a pulley support section (122) which supports the pulley (15), the pulley support section (122) being slidably connected to a middle section (121) of the main structure (12) so as to permit the distance between the pulley and the middle section (121) to be adjusted.

6. The framework as claimed in claim 5, wherein the pulley support section (122) is fixable in any one of a number of different discrete extended positions by means of a locking adjustment arrangement.

7. The framework as claimed in claim 6, wherein the locking adjustment arrangement comprises a plunger resiliently biased into engagement with any of a series of indentations provided in spaced apart arrangement on the pulley support section (122), to fix the pulley support section (122) in any of a plurality of predetermined positions.

8. The framework as claimed in claim 1, wherein the pulley (15) is elongate in an axial direction so as to provide a plurality of distinct pulley surfaces (15a, 15b, 15c), to allow traction to be applied along varying axes of the framework.

9. The framework as claimed in claim 1, wherein the main structure (12) and the leg structure (10) are foldably connected by means of at least one adjustable locking linkage (13), such that the leg structure (10) can be moved and locked into at least two different predetermined positions relative to the main structure (12).

10. The framework as claimed in claim 9, wherein the locking linkage (13) includes a boss (23), a locking screw (24) arranged to pass through a hole in the boss and operable in association with a locking nut (25) to hold the main structure (12) and leg structure (10) against one another, and wherein the boss (23) is shaped to provide at least two scalloped locating grooves (1, 2) for locating a portion of the main structure (12) or leg structure (10) relative to the boss (23) in order to define the predetermined relative positions.

11. The framework as claimed in claim 1, wherein the leg structure (10) further comprises a removably attachable support member (20) for supporting bed covers, in use.

12. The framework as claimed in claim 1, the framework being formed from generally tubular members.
